(19) United States
(12) Patent Application Publication
(10) Pub. No.: US 2006/0200038 A1
(43) Pub. Date: Sep. 7, 2006

(54) Noninvasive nonlinear systems and methods for predicting seizure

(76) Inventors: Robert Savit, Ann Arbor, MI (US); Ivo Drury, Ann Arbor, MI (US); Dingzhou Li, Carmel, IN (US); Weiping Zhou, Hanover, NH (US)

Correspondence Address: David A. Casmir;Medlen & Carroll, 101 Howard Street, Suite 350, San Francisco, CA, 94105, US

(21) Appl. No.: 10/527,781

(22) PCT Filed: Sep. 12, 2003
(86) PCT No.: PCT/US03/28700

§ 371 Date: Mar. 31, 2006

Related U.S. Application Data
(60) Provisional application No. 60/410,695, filed on Sep. 13, 2002.

(51) Int. Cl. A61B 5/04

(52) U.S. Cl. 600/544

(57) ABSTRACT

The present invention relates to methods and devices for noninvasive nonlinear prediction of ictal onset in patients afflicted by neurological disease. In particular, the present invention provides methods and devices for noninvasive nonlinear prediction of seizures in patients afflicted with epilepsy. The devices and methods preferably being based on analysis of two or more electroencephalogram (EEG) recordings, one set of recordings taken from an electrode close to the region of ictal onset, and a second or more set of recordings (e.g., concurrent readings) taken from a region remote from the region of ictal onset.

This invention claims priority to U.S. Provisional Application Ser. No. 60/410,695 filed on 13 Sep. 2002. The entire disclosure of the priority application is specifically incorporated herein by reference.

This invention was supported in part with grant R01 NS036803 from the National Institutes of Health. The United States government may have rights in this invention.

### FIELD OF THE INVENTION

The present invention relates to methods and devices for noninvasive nonlinear prediction of ictal onset in patients afflicted by neurological disease. In particular, the present invention provides methods and devices for noninvasive nonlinear prediction of seizures in patients afflicted with epilepsy. The devices and methods preferably being based on analysis of two or more electroencephalogram (EEG) recordings, one set of recordings taken from an electrode close to the region of ictal onset, and a second or more set of recordings (e.g., concurrent readings) taken from a region remote from the region of ictal onset.

### BACKGROUND OF THE INVENTION

Epilepsy affects between 40 and 50 million people worldwide—nearly 1% of the world's population. In the U.S. epilepsy affects about 2.5 million individuals at any given time with about 150,000-200,000 new cases diagnosed per year. (Begley et al., Epilepsia, 41:342-351 [2000]). Due to the persistent stigma surrounding neurological diseases such as epilepsy, the actual number of people affected by the disorder may actually be even higher.

In approximately 75% of affected individuals, the epilepsy has no identifiable cause. In the other 25% of afflicted individuals, the most common causes of epilepsy are brain injuries and trauma (e.g., hypoxia/anoxia, especially during birth), substance abuse, infections (e.g., meningitis), brain tumors or stroke, genetic defects, and degenerative disorders such as Alzheimer's disease.

An epileptic incident is characterized by intermittent interruption of normal brain function by sudden and often intense periods of synchronous neural discharge, resulting in either convulsive seizures, or more subtle alterations in neurological function such as brief lapses in full consciousness.

Epilepsies come in many forms and have many causes. A central distinction in the classification of epilepsies is between those with origins in an identifiable region of the brain, called focal or partial epilepsies (e.g., mesial temporal lobe epilepsy), and those with no well defined site of origin, called generalized epilepsies. Each of these two major categories may be subdivided into symptomatic (due to a known cause e.g., a brain tumor) type or idiopathic (of unknown cause but commonly suspected to be heredofamilial in origin) type. Symptomatic epilepsies are often medically intractable. A diagnosis of intractable epilepsy usually comes after two or three years of unsuccessful treatment with standard anti-seizure medications in a compliant patient.

While it is useful for neurologists to try to group epilepsies into categories for treatment, in reality, there can be significant overlap in the physical manifestations of a seizure disorder between patients with different types of epilepsy. One thing is clear, different epilepsies result from malfunctions in particular areas of the brain. Medicine has yet to provide well defined parameters for the different types of epilepsies, and has chosen instead to adopted a landscape classification approach in which various features such as clinical history, interictal (between seizures) EEG manifestation and results of neuroimaging (e.g, MRI) serve as useful landmarks. To date there is essentially no understanding of why a particular patient will have a seizure at any point in time. As a consequence, the prediction and treatment of epileptic seizures in individual patients remains very challenging.

The unpredictability of ictal (seizure) onset in an individual afflicted with epilepsy is perhaps the most difficult aspect of living with an epileptic condition. Many types of epileptic seizures totally incapacitate the afflicted individual for from moments to hours. Loss of one's facilities while controlling a vehicle or operating machinery, among many other things, can lead to potentially dangerous situations for the epileptic and for others at large.

What are needed are new portable and noninvasive methods and devices for reliably predicting ictal onset in a variety of patients. Such methods and devices will allow those suffering from many types of epilepsies heretofore unknown freedoms.

### SUMMARY OF THE INVENTION

The present invention relates to methods and devices for noninvasive nonlinear prediction of ictal onset in patients afflicted by neurological disorders. In particular, the present invention provides methods and devices for noninvasive nonlinear prediction of seizures in patients afflicted with epilepsy. The devices and methods preferably being based on analysis of two or more electroencephalogram (EEG) recordings, one set of recordings taken from an electrode close to the region of ictal onset, and a second or more set of recordings (e.g., concurrent readings) taken from a region remote from the region of ictal onset.

Embodiments of the present invention may be configured to predict the onset of ictal episodes in subjects having partial epilepsies including, but not limited to, benign occipital epilepsy (benign focal epilepsy with occipital paroxysms), benign rolandic epilepsy (benign focal epilepsy with centrotemporal spikes), frontal lobe epilepsy, occipital lobe epilepsy, mesial temporal lobe epilepsy, other forms of temporal lobe epilepsy, and parietal lobe epilepsy.

Broadly speaking, there are two types of epilepsy, focal and generalized. In focal epilepsies there is thought to be a specific region of the brain from which the seizures originate (although, as we shall discuss below, this notion is vague and the reality may be considerably more complex). The most common type of focal epilepsy is temporal lobe epilepsy (TLE), in which the region of ictal (seizure) onset is in one (rarely both) of the temporal lobes. Generalized epilepsies are those in which there is no clearly identifiable site of ictal onset.

Of all epilepsies, about 50% are focal epilepsies, and of these roughly 70% are epilepsies of the temporal lobe. Of patients with focal epilepsy, roughly 25% suffer a medically refractory condition, so that the only possible treatment currently available to them that might result in control of their seizures is surgical resection of part of the temporal lobe. For these patients, in particular, the present invention provides a reliable portable method for seizure prediction. The present invention allows the patient to reliably position himself in a safe environment (e.g., not driving, away from machinery, etc.) to weather the seizure. Additional embodiments of the present invention further incorporate one or more devices (e.g., electrical stimulation, medication dispensers, and the like) for administering therapies sufficient for aborting ictal onset or for lessening its effects.

While the present invention is principally directed to providing methods and devices for predicting the onset of epileptic seizures arising at a particular focal point in the brain, additional embodiments are optimized for predicting the onset of generalized idiopathic and/or symptomatic epilepsies. Generalized epilepsies usually are not localized to a focal point in the patient's brain as the patient's whole brain is affected by ictal episodes. As a result, generalized idiopathic epilepsies often produce more generalized symptoms in the patient. Examples of generalized idiopathic and/or symptomatic epilepsies include, but are not limited to, benign myoclonic epilepsy in infants, juvenile myoclonic epilepsy, childhood absence epilepsy, and juvenile absence epilepsy, infantile spasms (West syndrome), Lennox-Gastaut syndrome, and progressive myoclonus epilepsies.

In still further embodiments, the present invention provides systems and methods directed to predicting ictal onset in patients afflicted with unclassified and cryptogenic epilepsies such as febrile convulsions, Landau Kleffner syndrome, and Rasmussen's syndrome.

In some embodiments, the present invention provides a system for predicting ictal onset in a subject comprising: a first data sensor positioned on the scalp of a subject near the focal point of ictal onset; a second data sensor positioned on the scalp of the subject, wherein the second data sensor is remote from the first data sensor; and a processor configured to analyze data collected from the first and the second data sensors to provide a nonlinear mathematical manipulation of the data collected from the first and from the second data sensors, wherein the nonlinear mathematical manipulation produces a first marginal predictability value, and a second marginal predictability value. Preferably, the first and the second data sensors comprise electrodes. The present invention is not limited by the number, type, or placement of electrodes on a subject. In some embodiments, a plurality of electrodes (e.g., 2 . . . 10 . . . 100 . . . 200 . . . or more) may be placed about (e.g., implanted or attached (e.g., glued, taped, etc.) to the subject's skin). It is further contemplated that those skilled in the art of electrode placement can determine the suitable type, number, and placement of electrodes to best predict the seizures in a particular subject.

In preferred embodiments, electrodes are used to collect electroencephalogram (EEG) data from subjects. The present invention is not limited however to collecting EEG data from subjects. Indeed, other types of data may be collected and used to help predict seizures in a subject. For example, some embodiments of the present invention contemplate using, MEG, and/or ECoG data. In still further embodiments, the methods and systems of the present invention are configured to incorporate subject data collected from other sources such as magnetic resonance imaging (MRI), x-rays, including, computed tomographic (CT) scans, various genotypic and phenotypic based tests and observations, including, but not limited to, comparative genomic hybridization, polymerase chain reaction based microsatellite analysis, fluorescence in situ hybridization studies, tissue biopsies, physical examinations, and the like.

In preferred embodiments, the methods and systems of the present invention comprise a processor component (e.g., a computer comprising a computer readable memory and/or a devices to establish a communications link with one or more other component devices of the present systems) configured to compare/analyze the difference between a first marginal predictability value and a second, or more, marginal predictability value. In some embodiments, the marginal predictability values are computed by manipulation of subject data using nonlinear techniques and manipulations. However, the present invention is not limited to only using nonlinear data manipulation techniques. Indeed, in certain embodiments, one or more additional analysis techniques/values are considered, such as linear manipulations, calculation of various entropies, wavelets (i.e., portion(s) of the EEG with certain temporal and frequency characteristics) are analysis.

In particularly preferred embodiments, difference determined between a first marginal predictability value and the second, or more, marginal predictability value decreases as a seizure, or a set of seizures, in a subject approach thus indicting ictal onset.

Additionally, the present invention also provides methods and systems comprising one or more subject warning devices. In some of these embodiments, the subject warning devices are configured to receive data from a processor (e.g., computer processor) predicting that a seizure, or a set of seizures, in the subject is likely approaching. Accordingly, preferred embodiments of the present invention provide warning to the subject (or a third party, such as, medical professionals, first responders, caretakers, and the like) that a seizure, or a set of seizures, is likely approaching. A number of subject warning devices are contemplated including, but not limited to, audible, visual, and tactile alarms.

Some embodiments of the present invention further provide, systems comprising at least one component device in communication with a processor that administers anti-seizure agents to the subject. Anti-seizure agent administering devices suitable for use in the present, invention include, but are not limited to, drug delivery devices (e.g., pumps, micropumps, patches, and the like), and electrical stimuli generators, and the like. In some embodiments, the component devices (e.g., electrodes, component device for administering anti-seizure agents, processors, communications links, power supplies, etc.) are implanted in the subject; in some other embodiments, they are not implanted.

In yet other embodiments, the methods and systems of the present invention provide additional component devices and communications links to one or more third parties that transmit information from the system predictive of a seizure, or set of seizures, in the subject.

In additional embodiments, the present invention provides methods for predicting ictal onset in a subject comprising providing: a subject; a system configured to detect ictal onset, wherein the system comprises a first data sensor positioned on the scalp of the subject near the focal point of ictal onset; a second data sensor positioned on the scalp of the subject, wherein the second data sensor is remote from the first data sensor; a processor configured to analyze data collected from the first and the second data sensors to provide a nonlinear mathematical manipulation of the data collected from the first and from the second data sensors, wherein the nonlinear mathematical manipulation produces a first marginal predictability value, and a second marginal predictability value; and a subject warning device in communication with the processor; and contacting the subject with the system; determining the fist marginal predictability value and a second marginal predictability value; predicting ictal onset in the patient by difference in the fist marginal predictability value and a second marginal predictability value.

### DESCRIPTION OF THE FIGURES

FIG. 1 shows a schematic representation of some of the standard scalp (International 10-20 System) and sphenoidal electrode coverage over the left hemisphere. Right hemisphere electrodes match those displayed, but have the next highest even number.

FIG. 2A shows δ₂ as a function of time for a temporal electrode (F8) ipsilateral to the side of ictal onset during a 20 minute interictal period.

FIG. 2B shows δ₂ as a function of time for the occipital (O2) electrode ipsilateral to the side of ictal onset during the same period as in FIG. 2A.

FIG. 3A shows δ₂ as a function of time for a temporal electrode (F8) ipsilateral to the side of ictal onset during a one hour preictal period leading up to a seizure

FIG. 3B shows δ₂ as a function of time for the occipital (O2) electrode ipsilateral to the side of ictal onset during the same period as in FIG. 3A.

FIG. 4A shows Q₂ as a function of time during the same period as in FIG. 2A.

FIG. 4B shows Q₂ as a function of time during the same period as in FIG. 3A.

FIG. 5A shows δ₂ as a function of time for a temporal electrode (F7) of a non-epileptic subject during a 20 minute period.

FIG. 5B shows δ₂ as a function of time for the occipital electrode (O1) of a non-epileptic subject during the same period as in FIG. 5A.

FIG. 5C shows Q₂ as a function of time during the same period as in FIG. 5A.

FIG. 6 shows the sum of positive ranks (SPR) for a collection of 40 interictal epochs of 20 minute duration taken from 8 patients with TLE.

FIG. 7 shows the SPR test for 24 one hour preictal epochs prior to seizure onset from the same cohort of patients as in FIG. 6

FIG. 8 shows the SPR for 24 epochs of 20 minute duration taken from 6 non-epileptic subjects.

FIG. 9 shows Q₂ for 4 interictal and 3 preictal epochs for one patient.

### DEFINITIONS

To facilitate an understanding of the present invention, a number of terms and phrases are defined below.

As used herein, the term “epilepsy” refers to a heterogenous condition comprising recurrent unprovoked seizures characterized by abnormal transient paroxysmal disturbances (e.g., excitability) of cerebral cortical neurons, which leads to paroxysmal discharges in neuronal aggregates. Epileptic disturbances in brain function can manifest as episodic impairment or loss of consciousness, abnormal motor phenomena, psychic or sensory disturbances or perturbation of the autonomic nervous system. On the basis of origin, epilepsy is characterized as being either idiopathic (i.e., essential and/or genetic) or symptomatic (ie., acquired, secondary to a known insult such as a stroke or head trauma, or where the acquired cause is of uncertain origin [i.e., cryptogenic]). On the basis of clinical and electroencephalographic observations, four common subdivisions of epilepsy are recognized. One, idiopathic generalized, epilepsies of unknown cause but usually with a heredofamilial basis. Examples include, but are not limited to, juvenile absence, pure absence, and juvenile myoclonic epilepsy. Two, symptomatic generalized, epilepsies due to a know insult such as birth hypoxia or of uncertain insult (commonly referred to as cryptogenic). Examples include, but are not limited to, West Syndrome, and Lennox Gastaut Syndrome. Three, idiopathic localization-related (synonymous with idiopathic partial or focal), epilepsies believed to be of local origin in a part of the brain but likely due to heredofamilial causes. Examples include, but are not limited to, benign epilepsy of childhood with centrotemporal spikes (Benign Rolandic Epilepsy) and benign epilepsy of childhood with occipital spikes. Four, symptomatic localization-related (Symptomatic Partial or Focal), epilepsies commonly believed to be of local origin in the brain and due to an identified or cryptogenic insult. Examples include, but are not limited to, temporal lobe epilepsy, frontal lobe epilepsy, occipital, or parietal lobe epilepsy. Temporal lobe epilepsy is commonly subdivided into those seizures arising from mesial temporal lobe structures, and those from neocortical parts of the temporal lobe. Other subdivisions of epilepsy arising in other areas is also possible.

As used herein, the terms “seizure” or “ictal onset” refer to abnormal involuntary paroxysmal electrical activity (e.g., discharges of neurons) in the cerebral cortex that alters neurological function. Seizure can result in wide variety of clinical manifestations, including but not limited to, muscle twitches, staring, tongue biting, involuntary urination, loss of consciousness, and total body shaking.

As used herein, the term “subject” refers to an animal provided with the systems and methods of the present invention. Such organisms preferably include, but are not limited to, mammals (e.g., murines, simians, equines, bovines, porcines, canines, felines, and the like) and most preferably humans. In the context of the invention, the term “subject” generally refers to an individual having a neurological condition (e.g., epilepsy) contemplated to be benefited by use of the systems and methods of the present invention for seizure prediction.

The term “diagnosed,” as used herein, refers to the recognition of a disease by its signs and symptoms (e.g., uncontrollable neurological activity), genetic analysis, pathological analysis, or histological analysis, and the like. As used herein, the term, “in vitro” refers to an artificial environment and to processes or reactions that occur within an artificial environment. In vitro environments can consist of, but are not limited to, test tubes and cell cultures.

The term “in vivo” refers to the natural environment (e.g., an animal or a cell) and to processes or reactions that occur within a natural environment.

In preferred embodiments, the term “target cells” refers cells and tissues contemplated for use in the present invention. “Target cells” include, but are not limited to, neural cells.

As used herein, the term “marginal predictability” refers to the quantity defined in formula 6, below. It is a measure of the extent to which the dth lag in a time series or other data set provides additional predictive information for the next term in the series, give that the information in the intervening d-1 lags has already been used.

As used herein, the term “electroencephalograph (EEG),” and grammatical equivalents, relates to systems and devices for recording electric potentials of the brain derived from electrodes attached to a subject's scalp. Electroencephalographic equipment and techniques are used for collecting electroencephalographic data from a subject (e.g., a patient afflicted with epilepsy). This data is used by the systems and methods of the present invention to predict the onset of seizures (e.g., ictal onset) in the subject. The American Clinical Neurophysiology Society (ACNS, Bloomfield, Conn.) provides technical guidelines for performing EEGs.

The term “data sensors,” As used herein, refers to scalp based electrodes used for collecting data (e.g., EEG, EMG, and the like) from the subject that is subsequently used by the systems and methods of the present invention for predicting ictal onset in the subject.

As used herein, the term “intracranial data sensors,” refers to electrodes that are implanted in a subject's cranium used for collecting data (e.g., EEG, EMG, and the like) from the subject that is subsequently used by the systems and methods of the present invention for predicting ictal onset in the subject.

As used herein, the term “subdural data sensors,” refers to electrodes that are implanted under a subject's dura mater used for collecting data (e.g., EEG, EMG, and the like) from the subject that is subsequently used by the systems and methods of the present invention for predicting ictal onset in the subject.

As used herein, the terms “subject warning device,” or “patient warning device” refer to devices configured to receive information from a processor indicative of ictal onset and then to use this information to activate features of that device that alert a subject to the likelihood of ictal onset (e.g., a seizure). “Subject warning devices” suitable for use in the present invention include, but are not limited to, audible (e.g., buzzers, beepers, and the like), tactile (e.g., vibrating, pulsating, and the like), and visual alarms (e.g., flashing and the like).

As used herein, the term “preictal period” refers to the period of time preceding the onset of seizures (e.g., ictal onset) in a subject calculated by analysis of subject data (e.g., EEG, medical examination) that indicates that the subject is likely to start seizing in the near future (e.g., 1-5, 10, 30, 60, 120 min. or more). Likewise, the term “preictal warning period” refers to a portion (or all) of the “preictal period” where the subject is alerted to the likelihood of an ictal onset.

As used herein, the term “interictal” refers to the period of time between the termination of an isolated seizure or a set of seizures and the beginning of a second isolated seizure or set of seizures (e.g., ictal onset).

As used herein, the term “ictal onset” refers to the time or short epoch at which a seizure begins in a subject. The period of “ictal onset” is usually preceded by a “preictal period” of sufficient duration such that the systems and methods of the present invention provide the subject with a sufficient “preictal warning period” to begin administration of anti-seizure therapies and/or to prepare (e.g., stop/abstain from operating machinery etc.) for the onset of seizures.

As used herein, the term “focal point of ictal onset” refers to location(s) in a subject's brain where abnormal discharges (e.g., seizures) originate and spread forth from.

As used herein, the term “post ictal” refers to the period of time immediately following the cessation of seizures in a subject to a point in time when the subject has returned to baseline levels of neurological function.

As used herein, the term, “anti-seizure agent” refers to a compound or to an electrical stimulus used to treat epilepsy in a subject. Treatment is meant to encompass preventing, delaying, or lessening the severity of seizures and generally controlling epilepsy and its effects in a subject.

As used herein, the term “anti-seizure agent administering device,” refers to a device configured to administer at least one anti-seizure agent to a subject upon receiving instruction (e.g., via a communications link and/or user interface, etc.) provided by the systems of the present invention (e.g., a computer processor), the subject, a healthcare provider, a first responder, or a caretaker, etc.

As used herein, the term “effective amount” refers to the amount of an agent (e.g. an anti-seizure drug) or to the characteristics of the voltage of electrical stimuli sufficient to produce beneficial or desired effects in a subject (e.g., a subject having epilepsy). An effective amount of a drug or other therapeutic can be provided in one or more administrations, applications, or doses and is not intended to be limited to particular formulation or administration routes.

As used herein, the term “modulate” refers to the activity of an anti-seizure agent such as a medicament (e.g., an anti-seizure drug) or other therapeutic agent (e.g., electrical stimuli) to affect (e.g., to promote or retard) any aspect of neural cell function, including, but not limited to, electrochemical discharge and the like.

As used herein, the term “program,” when used as a noun, refers to an organized list of instructions that, when executed (i.e., performed by the computer) causes the computer to behave in a predetermined manner (e.g., to calculate and compare the difference in marginal predictability values for each of the two time series). Programs consist of modules, each of which contains one or more “routines/subroutines” (i.e., a section of a program that performs a particular task). In preferred embodiments, the programs described herein are written in a high-level programming language, such as BASIC, FORTRAN, C, C++, PASCAL, COBOL, or LISP, and the like.

As used herein, the terms “processor,” “computer processor,” “computer,” and “central processing unit,” or “CPU” are used interchangeably to refer to a device that is able to read a program from a computer memory (e.g., ROM or other computer memory) and perform a set of steps according to the program. The systems and methods of the present invention for seizure prediction are not intended to be limited, however, to implementation by processors. Indeed, in some embodiments the present invention contemplates systems comprising logic chips, logic arrays, gate arrays, application-specific integrated circuits, and programmable logic devices (PLDs), and the like.

As used herein, the terms “memory device” , “computer memory device”, or “computer memory” refer to any storage media readable by a computer processor. Examples of computer memory include, but are not limited to, RAM, ROM, computer chips, digital video disc (DVDs), compact discs (CDs), hard disk drives (HDDs), and magnetic tape.

As used herein, the term “computer readable medium” refers to any device or system for storing and providing information (e.g., data and instructions) to a computer processor. Examples of computer readable media include, but are not limited to, DVDs, CDs, HDDs, and magnetic tapes.

The term “operably linked,” in one sense, when used in reference to the operation of the disclosed systems and methods for predicting ictal onset refers to the execution (e.g., performance by a computer) of a computer program by a computer processor (or other suitable devices such as, logic chips and the like) to produce a desired result (e.g., the prediction of ictal onset in a subject). In another sense, the term “operably linked,” when used in reference to the operation of the disclosed systems and methods, refers to computer hardware devices and other apparatuses such as, communications links [e.g., fiber optics, modems, infrared [IR], LANs, the Internet]) configured to receive and/or exchange information with the disclosed systems, methods and programs for predicting ictal onset. In preferred embodiments, operably linked computer hardware devices and other apparatuses are configured to receive and/or exchange information with a computer program stored in computer readable memory associated with a computer processor via, for example, wires or cables, computer cards and boards, circuits, communication links [e.g., fiber optics, modems, IR, LANs, the Internet], etc., and any necessary device drives or computer program subroutines stored in computer readable memory.

As used herein, the terms “user,” and “system user” when used in reference to controlling the operation of a computer program (e.g., a software program stored in computer memory that predicts ictal onset), refers to a person, or a second computer program and system (e.g., a software program stored in computer memory), that controls the operation of the first computer program by selecting and/or entering system operation parameters and information. In some embodiments, the “user” is also the “subject.”

As used herein, the term “user interface” refers to the junction between a user and a computer program (e.g., the user interface is configured to be capable of receiving information from the user). A user interface allows the user to transmit or convey commands (e.g., instructions) to a computer program, hardware device or apparatus to perform specific tasks (e.g., prediction of ictal onset). “Graphical user interface,” or (GUI), refers to a user interface that takes advantage of the computer's graphics capabilities for entering and retrieving data from a program.

The term “Internet”, as used herein, refers to a collection of interconnected (public and/or private) networks that are linked together by a set of standard protocols (such as TCP/IP and HTTP) to form a global, distributed network. While this term is intended to refer to what is now commonly known as the Internet, it is also intended to encompass variations that may be made in the future, including changes and additions to existing standard protocols.

As used herein, the terms “World Wide Web” or “Web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP.

### DESCRIPTION OF THE INVENTION

Epilepsy is often though of as being a symptom of some underlying brain dysfunction that causes the dysfunction of brain cells, either at a specific place (as in focal epilepsy), or more widely (as in generalized epilepsy). This neurological dysfunction causes physical consequences such as the loss of consciousness or loss of muscle control.

Despite the difficulties in classifying different types of epilepsies the medical sciences have made several useful distinctions in the types of epilepsies. For example, partial epilepsies are characterized as originating from a clearly defined focal area within the brain. As a result, partial epilepsies have symptoms characteristic of their site of origin, such as simple visual hallucinations (for seizures of the occipital lobe) and unilateral motor difficulties (for seizures arising from the frontal lobe).

The present invention relates to methods and systems/devices for noninvasive nonlinear prediction of ictal onset in patients afflicted by neurological conditions. In particular, the present invention provides methods and systems for noninvasive nonlinear prediction of seizures in patients afflicted with epilepsy. In particularly preferred embodiments, the systems and methods of the present invention are directed to predicting seizures (e.g., ictal onset) in a subject afflicted with a focal (localization-related or partial) epilepsy.

The systems and methods preferably being based on analysis of two or more electroencephalogram (EEG) recordings, one set of recordings taken from an electrode close to the region of ictal onset, and a second or more set of recordings (e.g., concurrent recordings) taken from a region remote from the region of ictal onset. The respective recordings of subject data (e.g., EEG data) are each converted into time series. Either or both of the electrode locations may be ipsilateral to (i.e., on the same side as), or contralateral to (i.e., on the opposite side as) the focal point of seizure onset. In particularly preferred embodiments of the present invention, a comparison is made between the EEG data retrieved by a first electrode adjacent to the site of seizure onset (e.g., temporal location) and second electrode remote (e.g., occipital location) from the site of seizure onset. However, in some other embodiments of the present invention, different combinations and placements of electrodes are contemplated. In some embodiments, electrodes are placed about a subject following the standard 10-20 placement system. However, those skilled in the art appreciate that alternative electrode placements are preferable in recording data from certain types of epilepsy or other seizure causing disorders. Indeed, while collecting and manipulation of a subject's EEG data is preferable in some embodiments, collection and manipulation of other types of data are contemplated in additional embodiments (e.g., MEG, ECoG, and the like).

A nonlinear quantity called the marginal predictability (MT) is calculated for each of the time series generated using the formulas described herein. These MP values are then compared. It is contemplated that the difference between the two MP values predictably decreases several tens of minutes (e.g., 10, 20, 60 min., or more) prior to ictal onset. It is during a portion of the preictal period that the subject is preferably warned of the potential for an oncoming seizure (ictal onset) by one or more subject warning devices. In still other embodiments, the MP values (or other related quantities) are calculated based on intracranial or subdural data recordings.

The systems and methods of the present invention are not intended to be limited to incorporating any particular type of subject warning device(s). For example, in some embodiments, suitable warning devices for use with the seizure prediction systems and methods include, but are not limited to, audible (e.g., beeping devices), tactile (e.g., vibrating devices), visual warning devices (e.g., flashing devices). The present invention contemplates that one or more of these types (or other types) of warning device be incorporated into the systems and methods of the invention. In some embodiments, the subject warning devices provide the subject with an escalating warning signal (e.g., increasing volume audible alarm) as the onset of an ictal episode draws closer (e.g., temporally near).

In preferred embodiments, subject data is collected on one or more channels (e.g., 1, 2 . . . 4 . . . 8 . . . 16 . . . 32 . . . 128 . . . 256 . . . or more). In preferred embodiments, the foci of a subject's seizures are mapped, for example, using the International 10-20 system of electrode placement. In preferred embodiments, the number and placement electrodes (data sensors) used with a particular subject correspond to the determination of seizure foci. In particularly preferred embodiments, the systems and methods of the present invention collect patient data from two channels.

Some embodiments of the present invention use electrodes specially designed to reduce noise acquired from the data acquisition process (e.g., system noise, subject muscle contractions, etc.). For example, in one embodiment, the systems use one or more electrodes that integrate a first amplifier stage with a sintered Ag—AgCl electrode to provide extremely low-noise and virtually interference free extracranial data sensing (e.g., BioSemi, Amsterdam, Netherlands). In preferred embodiments, extracranial electrodes are used with noise levels near the thermal noise level of electrode impedance.

Preferably, the other components of the systems of the present invention are selected based on considerations, including, but not limited to, size (e.g., miniaturization), weight, power consumption, impedance, signal-to-noise ratio and the like.

In still further embodiments, subject data is transmitted via a communications link (e.g., telephone line, wireless network (e.g., radio wave, infra red, microwave, etc.), coaxial cable, fiber optic cable, wire, circuit path, and the like) to a processor configured to analyze the subject data and to determine the temporal proximity of an ictal episode. The communication links of the present invention are not intended, however, to be limited to any particular frequencies or wavelengths. Thus, in some embodiments, the processor is located remote from the subject. In other embodiments, subject data acquisition device(s) (e.g., EEG electrodes) are integral to the processor used. In preferred embodiments, all of the devices/systems and methods of the present invention are integrated and sufficiently miniaturized such that they are easily carried by the subject.

In additional preferred embodiments, the systems and methods of the present invention further incorporate data storage devices. It is contemplated that the data storage devices be configured to store the programs for predicting seizures from subject data. It is further contemplated that the data storage devices be configured to record subject data (e.g., EEG recordings) such that the data is transferable (e.g., via a communications link) to one or more additional devices. Data storage devices suitable for use in the systems of the present invention include, but are not limited to, floppy disks, hard disks, random access memory, digital tapes, compact disks, digital video disks, and the like. In preferred embodiments, subject data is periodically (e.g., in real time, hourly, daily, weekly, monthly, etc.) transferred to the subject's healthcare provider or to another third party (e.g., emergency responders, insurers, employers, and the like). As described above, subject data is transferred by any suitable communications link including encrypted or unencrypted transmission over the Internet. Thus, in certain embodiments, the systems and programs of the present invention for predicating ictal onset are accessible via the Internet (i.e., World Wide Web) over a communication network. Prior to transmission, transfer, or storage subject data is preferably compressed using one or more software compression programs.

Certain embodiments of the systems of the present invention further provide one or more component devices for administering therapeutic anti-seizure (e.g., drugs and/or electric stimuli) agents/treatments to the subject. In preferred embodiments, administrations of anti-seizure agents/treatments are timed to coincide with the system's prediction of ictal onset. In other embodiments, administrations of anti-seizure agents/treatments are at regular scheduled intervals (e.g., hourly, daily, weekly, etc.). Of course, additional variations on the administration times and routes of anti-seizure agents/treatments are within the scope of the present invention. For example, in some additional embodiments, component devices are configured to administer anti-seizure agents on demand. In still other embodiments, a command to the anti-seizure agent administering device instructing it to administer the anti-seizure agent is sent from a third device (e.g., a processor) or entity remote from the seizure prediction systems via a communications link (e.g., telemetry link). In yet other embodiments, the seizure prediction systems of the present invention optionally incorporate additional programs and devices for determining the proper time(s) to administer anti-seizure agents to increase their effectiveness.

The present invention further contemplates a number of component devices that re suitable for administering anti-seizure agents to subjects. For example, in some embodiments, the systems of the present invention incorporate electric stimulus devices (e.g., microelectromechanical systems (MEMS), or wireless integrated microsystems devices (WIMS), and the like) configured to provide the subject with mild electrical pulses prior to ictal onset. For example, in some embodiments, the present systems incorporate devices configured to provide the subject with adaptive electric field therapy or vagus nerve stimulation (VNS) therapy (Cyberonics, Inc., Houston, Tex.). In some embodiments, the component devices for delivering electrical stimuli are implanted in the subject. In other embodiments, component devices for delivering electrical stimuli are not implanted in the subject. In particularly preferred embodiments, these devices are connected via a communications link the seizure prediction systems of the present invention. Optionally, these devices are also connected via a communications link to remote processors controlled by the subject's healthcare provider and the like.

Those skilled in the art appreciate that a number of implantable medical devices capable of delivering electric stimuli to a subject are suitable for use in the present systems (e.g., micro-electro-mechanical systems (MEMS)). The present invention is specifically not intended, however, to be limited to incorporation of implantable devices.

A number of systems for powering and controlling implantable/non-implantable medical devices are suitable for in the present invention. For example, some implantable component devices suitable for use in the present invention incorporate wireless power transfer systems that operate through an inductive link to a battery (e.g., rechargeable lithium battery) using bi-directional data communication. In other embodiments, implantable components such as micro pumps (actuators) and/or electric stimulators are configured to run on integral batteries. In particularly preferred embodiments, the batteries are energized using an RF antenna or low-frequency magnetic loop implanted in the subcutaneous tissue.

In still further preferred embodiments, the components of the present invention implanted in a subject are hermetically sealed and constructed of non-immunogenic materials.

Anti-seizure drugs suitable for use in some embodiments of the present invention include, but are not limited to, phenobarbitol, phenytoin, carbamazepine, ethosuximide, valproate, benzodiazepines (e.g., clonazepam, diazepam, and lorazepam), felbamate, gabapentin, lamotrigine, vigabatrin, topiramate, and the like. In preferred embodiments, one or more anti-seizure drugs are administered to a patient by a component device under the instruction of the systems of the present invention. For example, in a preferred embodiment, when the systems of the present invention detect ictal onset is likely, the system instructs (e.g., via a communications link) a component device (e.g., a micropump) to administer a therapeutically effective amount of an anti-seizure drug to the subject. In other embodiments, the systems of the present invention periodically (or aperiodically) instructs a component device (e.g., micropump) to administer a therapeutically effective amount of an anti-seizure drug based on a preset schedule (e.g., hourly, daily, weekly, etc.) or upon the subject's self instruction, or upon being instructed to do so by a third party (e.g., the subjects health care provider), or upon being instructed to do so by the invention which may instruct intervention in an aperiodic, or therapeutically random fashion. In some of these embodiments, the third party health care provider is remote from the subject and instructs a component device to administer an anti-seizure drug to the subject via a communications link.

In particularly preferred embodiments, the present invention provides methods and systems for predicting the onset of seizures in a subject suffering from a focal epilepsy (e.g., mesio-basal temporal lobe epilepsy). However, additional embodiments are provided for predicting the onset of seizures in subjects with various types of non-focal epilepsies.

In preferred embodiments, subject data tapes are collected from scalp-sphenoidal monitoring (e.g., data sensors) of epilepsy patients. Preferably, the subject data (e.g., EEG) is sampled at 200 Hz and the data tapes are read on a Viewmaster 5000 machine (BMSI/Nicolet, Madison, Wis.). The subject data is transferred via a communications link to a computer memory for storage. From the computer memory the data is transferred by via a communications link (e.g., ftp) to a computer workstation (e.g., Linux workstation). The subject data is compressed, such that each data point takes up about 2 bytes. In some embodiments, for purposes of analysis, the compressed subject data is extracted and converted from binary to ASCII using standard processes. Further data analysis is carried out on the computer workstation. In some embodiments, the subject data set used in the analyses of the present invention are decimated, and consist of every third data point from the original 200 Hz sampled data record. However, the decimation frequency is a parameter that can be changed in the analysis. Additional filtering of the original subject data is typically not necessary, however, in certain other embodiments original subject data is subjected to additional or different filtering steps.

Preferred embodiments of the systems and methods of the present invention comprise ambulatory devices in which one or more data sensors (e.g., electrode leads) are attached to the subject's scalp. The data collected from the several leads (e.g., EEG data) is temporarily stored for form one to several, or more, minutes in one or more temporary storage devices (e.g., computer memory) worn by the subject. These devices also contain a processor to compute the nonlinear and other quantities of interest. In some embodiments, once the data has been used for the calculations of interest, that data is purged and replaced with new subject data coming in real time from the subject's data sensors (e.g., electrodes). In other embodiments, the data is stored for longer periods of time and used in subsequent analyses in one or more temporary devices worn by the subject. The present invention is not limited however to storage devices, or other components, of the present invention that are worn by the subject. In still other embodiments, the subject data is retained and subsequently transferred via a communications link to additional storage devices and processors. In some of these embodiments, the subject data is optionally further compressed for easier transfer and storage. The computed value of the nonlinear (and possibly linear) quantities of interest are added to an existing record and are used with recently analyzed/manipulated (e.g., about the past 1 . . . 5 . . . 10 . . . 30 . . . or 60 min. or more) subject data to generate seizure predictions. In preferred embodiments, the time necessary to perform these calculations is such that new values of the nonlinear metrics are computed with only a few (e.g., 1 . . . 5 . . . 10 . . . to about 20) seconds lag from real time.

In some embodiments, the seizure prediction systems are attached physically, electronically, or by wireless communication to other devices(s) that provide medical or electrical intervention to abort a seizure or to ameliorate its effects.

Additional exemplary systems and methods of the present invention are described in more detail in the following sections: I. Mathematical considerations; and II. Data analysis.

I. Mathematical Considerations

The first attempts at seizure prediction relied on standard linear statistical methods. (See e.g., J. Gotman et al., Long-term monitoring in epilepsy, EEG suppl. No. 37 Amsterdam: Elsevier [1985], and I. Osorio et al., Epilepsia, 39:615-627 [1998]). Recent efforts by brain researchers have included finding nonlinearity and claims of chaos in brain study data sets (e.g., EEGs). (See e.g., J. Theiler, Phys. Lett. A., 196:335-341 [1995]; A. Babloyantz and A. Destexhe, Proc. Natl. Acad. Sci. U.S.A., 83:3513-3517 [1986]; G. W. Frank et al., Physica D, 46:427-438 [1990]). However, many attempts at applying nonlinear dynamics and chaos theory to biological systems have met with limited success. (See e.g., Theiler supra; J. Theiler and P. Rapp, Electroenceph. Clin. Neurophysiol., 98:213-222 [1996]; M. Palus, Nonlinearity in normal human EEG: Cycles, temporal asymmetry, non-stationarity and randomness, not chaos, Santa Fe Institute working paper 94-10-054 [1994]); Babloyantz and Destexhe supra, and L. D. Iasemidis et al., Brain Topog. 2:187 [1990]; A. Destexhe and A. Babloyantz, Deterministic chaos in a model of the thalamo-cortical system. In: Self-Organization, Emerging Properties and Learning, A. Babloyantz ed., Plenum Press, New York, N.Y. [1991]). Thus, prior to the present invention effective methods and systems for predicting ictal onset in subjects using noninvasive nonlinear analyses were unknown. For instance, most of the research concerning prediction of epileptic seizures has focused on collecting and analyzing intracranial data recordings. Intracranial data recordings have traditionally been available from a subset of epilepsy patients with medically refractory temporal lobe epilepsy (TLE). In contrast, however, preferred embodiments of the present invention collect and analyze subject data obtained from extracranial (e.g., scalp) electrodes.

However, in some other embodiments, the present invention contemplates placement of intracranial electrodes (e.g., in cases where the seizure focus is known).

In preferred embodiments of the present invention, nonlinear mathematical methods are used to manipulate subject data to predict the onset of seizures in an epilipetic subject. The basis for the nonlinear dynamical analyses used in the present invention is the correlation integral (P. Grassberger and I. Procaccia, Physica D, 9:189-208 [1983]) defined as:
C_{d}(y(i), y(j))=P(∥y^{(d)}(i)−y^{(d)}(j)∥<ε) (Formula 1)
where P(·) denotes the probability of the argument, xⱼ is the j^{th} element of the time series being reconstructed, and y^{(d)}(i)=(xᵢ, xᵢ₋₁, . . . , x_{i-d+1}) is a d-dimensional vector reconstructed from data. The notation ∥·∥ means norm of the argument. In preferred embodiments, the methods of the present invention use the max norm equation, which is the computationally simplest definition, i.e., ∥y^{(d)}(i)−y^{(d)}(j)∥<ε if max[|xᵢ₋ₖ−xⱼ₋ₖ|]<ε for k=0, 1, . . . , d-1. The present invention is not intended, however, to be limited to using this definition of norm. Indeed, a number of different definitions can be used for the norm. The skilled artisan will recognize that good theoretical and/or computational reasons for choosing one definition over the other exist in particular embodiments. Thus additional embodiments of the present invention use other definitions for norm. The quantity C_{d} is the probability that two vectors reconstructed from the time series in d-dimensions will be close to each other. In terms of the original time series, C_{d} is a measure of the likelihood that two sequences of length d taken from a time series will look similar. Using the C_{d}'s, predictability can be defined as (See, R. Savit and M. Green, Physica D, 50:95 [1991]):

$${S}_{d} = \frac{{C}_{d + 1}}{{C}_{d}} . \qquad ( Formula 2 )$$

In view of formula 1, S_{d} is the conditional probability equation
S_{d}=P(z_{d+1}|z_{d}, . . . , z₁) (Formula 3)
where,
zₖ=|xᵢ₊ₖ₋₁−xⱼ₊ₖ₋₁|<ε. (Formula 4)
Thus, in preferred embodiments, S_{d} is the conditional probability that if two randomly chosen d-tuples from the time series have their first d-1 elements within δ of each other, respectively, then the d^{th} elements will also be within δ.

In some embodiments, S_{d} is used as a nonlinear statistic. However, in preferred embodiments, a more sensitive discriminator of nonlinear structure in time series (See, R. Manuca et al., Mathematical Biosciences 147:1 [1996]); and R. Savit and M. Green, Physica D, 50:95 [1991]) is the ratio of S_{d}'s, defined as:

$${R}_{d} = \frac{{S}_{d}}{{S}_{d - 1}} = \frac{{C}_{d + 1} {C}_{d - 1}}{{C}_{d}^{2}} . \qquad ( Formula 5 )$$

To make the interpretation simple, preferred embodiments of the present invention define marginal predictability as:

$${δ}_{d} ≡ \frac{{R}_{d} - 1}{{R}_{d}} \qquad ( Formula 6 )$$

Wherein, δ_{d} is a measure of how much additional predictive information there is in the (d+1)^{st} lag of the time series, given that information in the intervening d lags has already been used. It is contemplated that if δ_{d} is close to zero, there is no additional predictive information, on average, for the current value of the time series in the value of the (d+1)^{st} lag. However, if δ_{d} is significantly different from zero, then S_{d}>S_{d-1}, and there is additional predictive information in the (d+1)^{st} lag. “Predictive information” here is understood in the sense of nonlinear dynamics. (See e.g., R. Savit and M. Green supra; and K. Wu et al., Physica D, 69:172 [1993]).

In particularly preferred embodiments, the methods of the present invention comprise comparing δ_{d} for two different scalp electrodes as a function of time. Thus, consider Q_{d}(A,B;t)=δ_{d}(A;t)−δ_{d}(B;t), where A and B are two electrodes and t is time. In preferred embodiments, A will be one or more electrodes (e.g., scalp electrodes) near the seizure focus and B will be an electrode (e.g., scalp electrodes) remote from and preferably ipsilateral to the site of ictal onset. In the case of TLE, B will generally be an occipital electrode. Thus, preferred embodiments of the present invention compare differences in the marginal predictabilities of temporal and occipital electrodes between times far removed from a seizure and times close to a seizure. In further embodiments, of the present invention the methods are based on similar calculations where i) A and B are contralateral temporal and occipital electrodes, respectively, and ii) A and B are temporal ipsilateral and contralateral electrodes, respectively.

In analyses based on non-linear dynamics, several parameters (e.g., embedding dimension, and tolerance) need to be set prior to performing the calculations for determining the MP values. Thus, in some embodiments, the basic seizure prediction methods of the present invention can be tailored to the needs of a particular patient or for predicting particular types of epileptic seizures by adjusting the values of the parameters (e.g., altering the dimension of the reconstruction space) of the nonlinear mathematical manipulations.

The present invention further contemplates additional embodiments wherein the methods of seizure prediction use one or more alternative mathematical quantities to increase the predictive value and robustness of the basic seizure prediction method in certain patients. For example, in some embodiments calculation of various entropies are contemplated. In other embodiments, wavelets (i.e., portion(s) of the EEG with certain temporal and frequency characteristics) are analyzed to further predict seizures. In still other embodiments, additional linear or nonlinear quantities may be used alone or in conjunction with MP to increase the selectivity and sensitivity of seizure prediction.

II. Data Analysis

In preferred embodiments, patient results are presented in 40 second windows comprising about 8000 data points (sampling rate of 200 Hz) and are used to calculate δ_{d} and Q [symbol] (A.B;t). Preferably, the time series xᵢ is obtained from the originally sampled EEG recordings by using every third data point (t=3). This decimation of the data set was chosen to minimize the value of the mutual information. (See, H. Tong, Nonlinear Time Series Analysis: A Dynamical Systems Approach, Oxford University Press [1990]). ε in (Formula 4) is 10% of the standard deviation of the time series, xᵢ. In other embodiments, it is contemplated that these values of τ and ε can be altered to improve predictability performance. Those skilled in the art appreciate the steps to alter this value without more than routine experimentation. In the results presented below, d=2. The results include twenty-four 20 minute preictal epochs from eight patients and forty interictal epochs of 20 minute duration from eight patients in the analysis. Behavior states of the subjects were noted and epochs were chosen that represented a range of behavior states including various stages of awake alertness and various sleep states. Results of the analysis were independent of the behavior state of the subject. For purposes of comparison, twenty four epochs of 20 minute duration from six non-epileptic subjects using methods of the present invention were also analyzed.

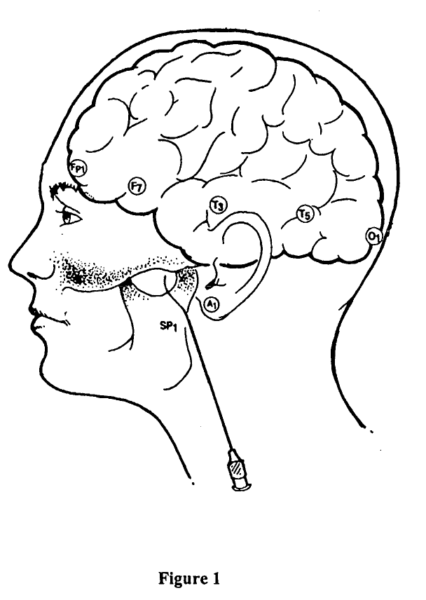

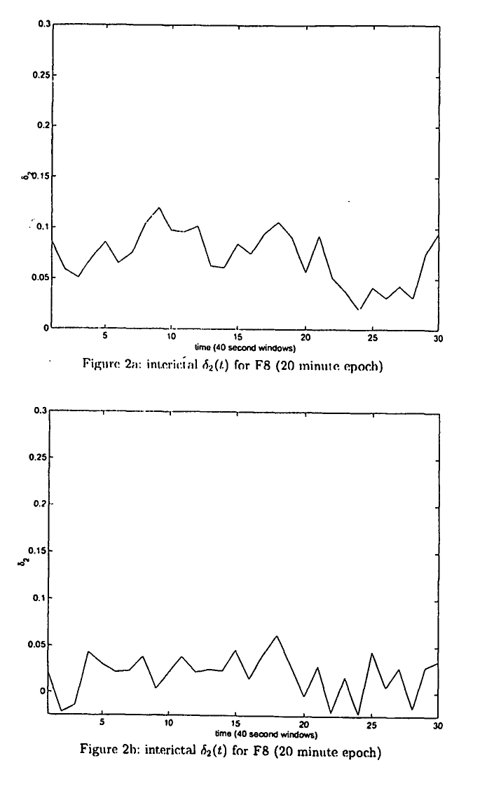

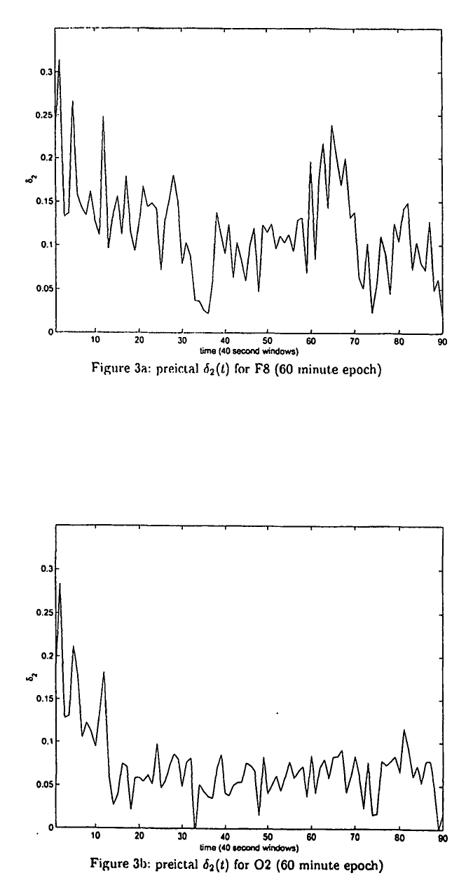

FIG. 1 shows a schematic diagram indicating the standard nomenclature and placement of scalp electrodes according to the International 10-20 System. In particular, FIG. 1 shows the left side of a hypothetical patient's head. Electrodes placed in homologous locations on the right side of the scalp are labeled with the next highest even number. Thus, for example, the electrode on the right side of the head in the position homologous to the F7 electrode is labeled F8. Depending on the side of the seizure focus, the F7 or F8 electrode is typically close to the site of ictal onset in cases of TLE. Two examples of δ₂ as a function of time are shown in FIGS. 2A-B and 3A-B, respectively. FIG. 2A shows the time course of δ₂ for a temporal electrode (F8) ipsilateral to the side of ictal onset during a 20 minute interictal period. FIG. 2B shows δ₂ for the occipital (O2) electrode ipsilateral to the side of ictal onset during the same period. δ₂(F8) is significantly higher than the δ₂(O2). FIG. 3A shows δ₂ for the same electrode (F8) used in FIG. 2A, but calculated during a one hour preictal period leading up to a seizure, and FIG. 3B shows δ₂ for the electrode (O2) used in FIG. 2B, calculated during the same one hour preictal period used for FIG. 2A. Note that δ₂(F8) is still greater than δ₂(O2) until about 15 minutes before the seizure onset. Within 15 minutes prior to the seizure, however, δ₂(F8) decreases to approximately the same level as δ₂(O2).

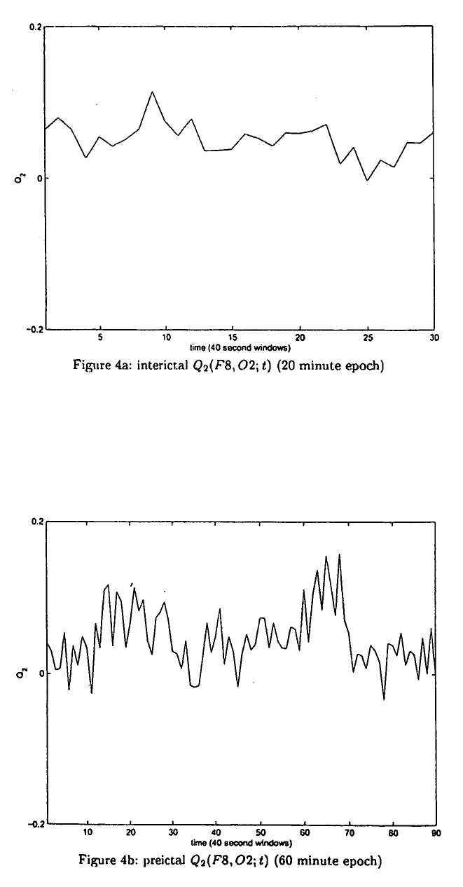

Certain embodiments of the present invention are illustrated by considering Q₂, which is the difference between the δ₂'s of different electrodes. The results are presented in FIGS. 4A-B. Note that in FIG. 4A, Q₂ is greater than zero for the interictal epoch (typically close to 0.05). For most of the early portion of the preictal epoch Q₂ is also greater than zero in FIG. 4B, but moves close to and stays near zero starting about 15 minutes prior to the seizure. Although there are differences in the profiles of the δ₂'s for different epochs, the features illustrated in FIGS. 4A an 4B, namely, the fact that Q₂ is smaller in the preictal compared to the interictal period can be found in almost all of the epochs studied from the epileptic subjects.

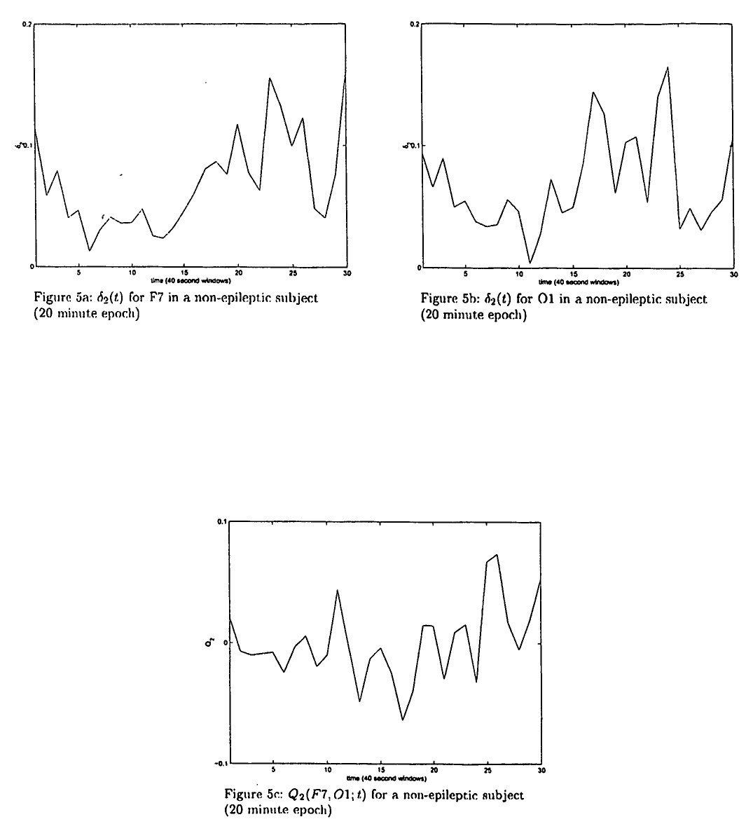

The same analysis was also applied to a set of epochs taken from non-epileptic subjects. FIGS. 5A and 5C are examples of a 20 minute epoch from a non-epileptic subject. In some of these embodiments, the electrodes comprise a temporal electrode (F7) and an occipital electrode (O1), both on the left hemisphere. FIG. 5C shows that the value of Q₂ for non-epileptic subjects are typically close to zero, which suggests that there is no systematic difference between the δ₂ of temporal electrodes and that of occipital electrodes for non-epileptic subjects.

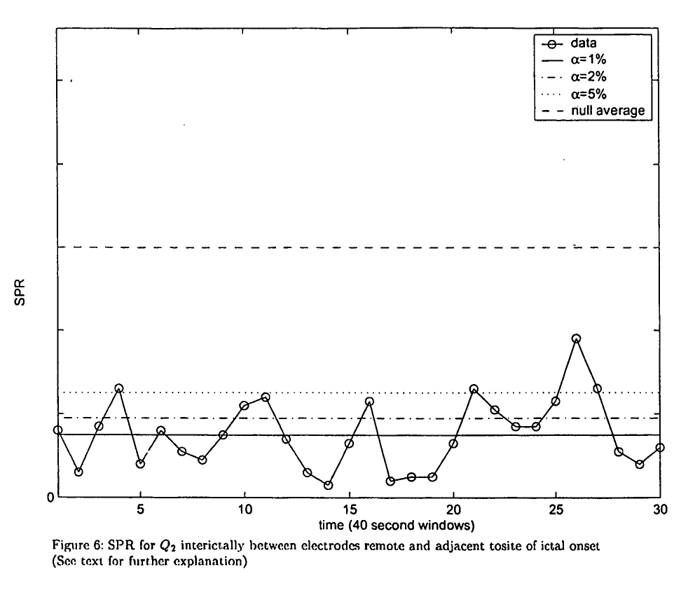

In order to statistically validate these observations, a Wilcoxon's sum of signed rank test was applied to the values of Q₂. However, the present invention is not limited to the Wilcoxon's sum of signed rank test. Those skilled in the art recognize that other nonparametric tests for manipulating paired samples are also useful in certain embodiments of the present invention. Briefly, the Wilcoxon test (See e.g., E. L. Lehmann, Nonparametrics: Statistical Methods Based on Ranks, Holden-Day, Inc., Oakland, Calif. [1975]) is a nonparametric test for paired samples (Xᵢ, Yᵢ). Accordingly, in the present methods the paired samples are the marginal predictabilities for focal electrodes and remote electrodes, respectively, i.e., (Xᵢ, Yᵢ)=(δ₂(F electrodes), δ₂(O electrodes). The Wilcoxon sum of signed rank test can be used to test the null hypothesis that the median of the difference, Dᵢ=Xᵢ−Yᵢ, is equal to zero, so that it is just as likely that Xᵢ>Yᵢ as that Xᵢ<Yᵢ. Specifically, the test is calculated in the following way: 1) rank order the absolute values of the Dᵢ from smallest to largest (wherein Rᵢ is the rank of |Dᵢ|, for i=1, . . . , n); 2) assign the sign of Dᵢ to the rank of Dᵢ; and 3) calculate SPR, (the sum of all the positive ranks), that is the sum of all those ranks that are associated with a positive value of Dᵢ. If the above null hypothesis, namely that the median of the differences, Dᵢ, is zero, is true, about half the Dᵢ values are positive and half negative, and SPR will be neither tool large nor too small, being close to n(n+1)/4, where n is the sample size. A test statistic can therefore be developed based upon SPR. Under the null hypothesis, the expected value of SPR is equal to n(n+1)/4, which is indicated by the broken lines in the FIGS. 6, 7, and 8. In preferred embodiments, the probability of each distinct value of SPR under the null hypothesis may also be calculated, giving significance levels. For example, for n=24, the probability that SPR is less than 81 under the null hypothesis is approximately 0.025. Hence, if SPR is less than 81, then rejection of the null hypothesis can be made with 97.5% confidence.

The advantage of nonparametric test (e.g., the Wilcoxon signed rank test) over parametric tests (e.g., t-test) is that it does not make any ancillary assumptions about the distribution of Dᵢ. The only necessary assumption is that all Dᵢ's are independently sampled from the same distribution.

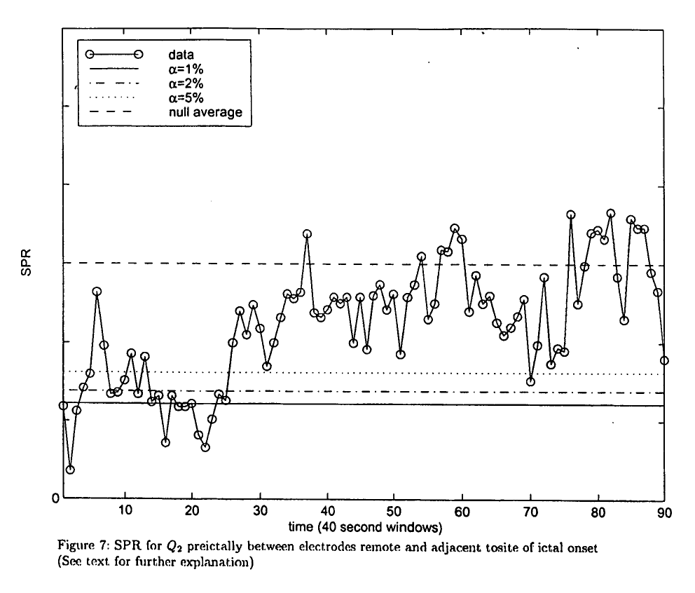

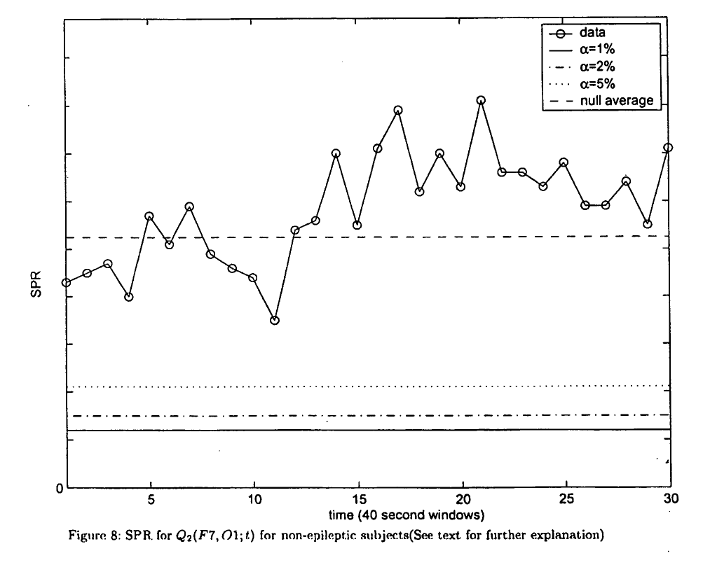

Specifically, summed-rank tests were performed to test the null hypothesis that the median of δ₂ of the electrode adjacent to seizure onset, μ_{adj} (e.g., electrodes F7 or F8, depending on which side of the brain contained the site of ictal onset), is the same as that of the electrode remote to the site of seizure onset μᵣₑₘₒₜₑ (e.g., occipital electrode O1 or O2 ipsilateral to the site of ictal onset), thus Q₂=0, statistically. The sum of positive ranks (SPR) as a function of time are shown in FIGS. 6, 7 and 8 for interictal, preictal and non-epileptic subjects, respectively. If SPR is close to the average under the null hypothesis (the broken line in the middle of the graph), then the null hypothesis cannot be rejected. However, if SPR, is too low, the null hypothesis must be rejected and the alternative hypothesis that μ_{adj}>μᵣₑₘₒₜₑ (i.e., Q₂>0) must be accepted. FIG. 6 shows the SPR for a collection of 40 interictal epochs of 20 minute duration taken from 8 patients with TLE. Each point represents one 40 second window. FIG. 7 is the SPR test for 24 one hour preictal epochs prior to seizure onset from this same cohort of patients, and FIG. 8 is the SPR for 24 epochs of 20 minute duration taken from 6 non-epileptic subjects.

FIG. 6 shows that the null hypothesis that the δ₂'s are the same for the adjacent and remote electrodes interictally must be rejected and the alternative hypothesis that Q₂>0 must be accepted. FIG. 7 shows that up to about forty minutes prior to seizure onset, the δ₂'s for the adjacent electrodes are significantly greater than those for the ipsilateral remote and the null hypothesis is below 5% significance level over that time period. Within about 40 minutes prior to seizure onset, the SPR increases making rejection of the null hypothesis no longer possible. The marginal predictability of the electrodes adjacent to the site of ictal onset is significantly greater than that of the ipsilateral occipital electrodes, except within about half an hour prior to a seizure, at which time the marginal predictabilities take on similar values.

Similar analyses comparing sphenoidal and occipital scalp electrodes have been preformed. The sphenoidal electrodes are also relatively close to the site of ictal onset in TLE. The results are qualitatively similar to those in FIGS. 5 and 6, but are somewhat less distinct, probably due to increased noise and artifact from the sphenoidal electrodes. By comparison, FIG. 8 shows the summed-rank test for epochs derived from non-epileptic subjects. In marked contrast to the interictal epochs in patients with epilepsy, FIG. 8 shows that for non-epileptic subjects, the null hypothesis cannot be rejected, and the δ₂'s for the ipsilateral temporal and occipital electrodes are statistically the same.

The invention contemplates that the statistical results obtained using the present methods are not related to the behavior state of the epochs in the set. For example, test were run on the subsets of the preictal set and the interictal data for which the subjects were asleep during all the epochs, qualitatively, the predictive value of the present methods and systems was unchanged.

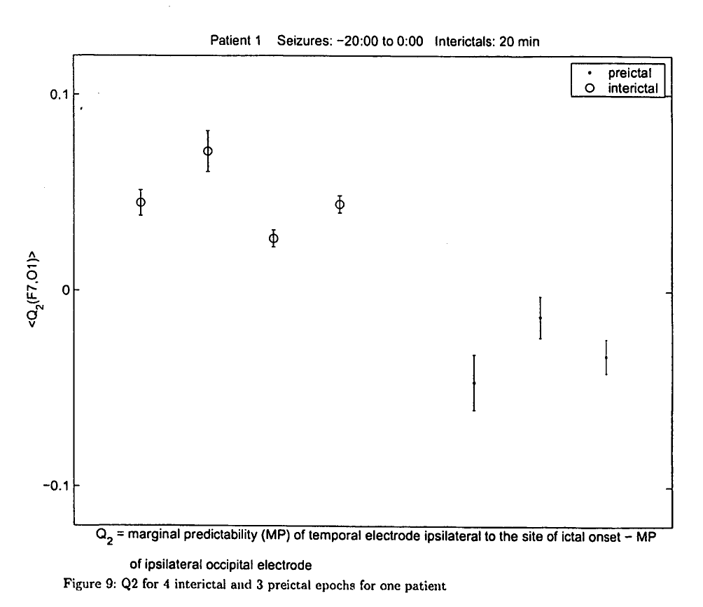

Additional studies indicate that the results described in the previous paragraphs can be disaggregated and that the same effect can be seen in results from individual patients. For example, FIG. 9 shows show the values of Q₂ for 4 interictal and 3 preictal epochs for one patient. It is clear that there is a systematic difference between the values of Q₂ obtained for interictal as opposed to preictal epochs. In fact, a survey of the individual results for the eight patients studied reveals that, although for each given patient, Q₂ during the preictal epoch is not necessarily statistically zero, it is always smaller than the typical values of Q₂ during the interictal epochs for that patient.

The patient results obtained from the methods and systems of the present invention strongly indicate a systematic change in a nonlinear measure computed on scalp EEG recordings prior to ictal onset in patients with epilepsy (e.g., medically refractory temporal lobe epilepsy).

### EXAMPLES

The following examples are provided to demonstrate and further illustrate certain preferred embodiments of the present invention and are not to be construed as limiting the scope thereof.

### Example 1

### Subject Selection

This example describes the selection processes used for selecting patients suitable for studies used to validate the methods of the present invention. Patients were evaluated by epileptologists at Henry Ford Hospital in Detroit. Presurgical evaluations followed a standardized protocol (A. M. Valachovic et al., Language and its management in the surgical epilepsy patient in Medical Speech-Language Pathology, A. F. Johnson and B. H. Jacobson eds. Thieme, New York, N.Y., pp. 425-466 [1998]). In order to provide a homogenous group of patients, selection was limited to those afflicted with medically refractory mesiobasal temporal lobe epilepsy. Patients with this type of epilepsy are generally regarding as the most suitable candidates for epilepsy surgery.

The specific criteria for inclusion in our analysis are: 1) seizures had to be of unilateral mesiobasal temporal lobe origin, documented by history, and interictal and ictal EEG recordings; 2) patients had to be between 18 and 60 years to reduce the likelihood of age related disorders such as cerebrovascular disease; 3) no mass lesions detectable with magnetic resonance imaging (MRI); 4) intelligence quotient of 70 or more; 5) no evidence of progressive neurological disorders, active neurological disorders other than epilepsy, and no other significant medical disorder, severe depression or psychosis; 6) no evidence of damage to the hippocampus contralateral to the seizure focus as determined by MRI; 7) no history of substance abuse; 8) patients receiving barbiturates or benzodiazepines were excluded with the exception of intravenous benzodiazepines used for acute seizure control; and 9) no history of drug use other than antiepileptic drugs during the two weeks prior to the recordings.

### Example 2

### Electroencephalogram (EEG) Recordings

Patient EEG recordings were recorded on a 128-channnel BMSI/Nicolet 5000 System. (Nicolet Biomedical, Madison, Wis.). The band pass is 0.5 Hz to 100 Hz. The digital data is then transferred to a Linux workstation for conversion to ASCII text data and further analysis. An experienced epileptologist and a clinical neurophysiologist reviewed all EEG recordings. EEG recordings from the patients were visually inspected to identify epochs of interest for analysis. Epochs were divided into the following sets: 1) interictal, meaning at least 1 hour before and at least one hour after a seizure; 2) preictal, meaning within the hour preceding a seizure, and at least 1 hour following a seizure; and 3) ictal. Epochs were separated by behavioral state into: 1) wakefulness; 2) drowsiness; 3) stage 2 non-REM sleep; 3) slow wave sleep; and 4) REM sleep. Waking and sleeping EEG from normal age and sex-matched subjects were analyzed.

### Example 3

### Study of MP and Different Behavior States

In the following example an experienced epileptologist reviewed the complete scalp EEG (26 channels) for 61 interictal and 33 preictal epochs, each 20 minutes long from 14 patients. The epileptologist categorized patient behavior during a plurality of 30 second interval of the epochs, and placed each interval into one of the following categories:

Awake, eyes open—AEO

Awake, eyes closed—AEC

Lightly drowsy—D1

Heavily drowsy—D2

Stage 2 NonREM sleep—S2

Stage 3 and 4 of NonREM sleep—S3/4

REM sleep—REM

From the set of 40 thirty-second intervals for a given epoch, a summary behavior score for that epoch was produced. If 32 or more of the 30 second intervals (80%) of a given epoch were in the same behavior state, then that 20 minute epoch was deemed primarily in that behavior state (e.g., AEO or D2). If 60-79% of an epoch was spent in one state, then that epoch was considered as predominantly that state and indicated with the prefix P, thus PAEO or PD2. When less than 60% of an epoch was spent in just one state, then the epoch was considered a blend of 2 or more states, thus AEO/D2. Listed below are the numbers of various behavior states that comprise the basic data set of interictal and preictal epochs.

AEO—49

PAEO—3

D2—9

S2—17

Mixed states—16

No states were observed that were purely or predominantly D1, AEC, S3/4 or REM. However, the states D1 and AEC do contribute to some of the mixed states.

The data was used to test for the dependence of <Q₂>, the value of Q₂ averaged over 20 minute epochs, on behavior state as well as on whether the epoch in question is preictal or interictal, and on whether the seizure focus is on the left or right side of the brain. The same methods were also used to test for the dependence of <δ₂> and the marginal predictabilities for channels both remote from and adjacent to the seizure focus on the same set of variables. As the models become more complicated the more behavior state variables are included. For behavior states for which there are few observations, it is not advisable to include additional variables. There are two approaches to this issue. The first approach is to agglomerate related behavior states (e.g., AEO and AEC) into one category. The second approach is to eliminate those states with few observations. Two separate analyses were done using each of these approaches. In both cases, the number of behavior states against which the invention tested for dependence in either case was four (e.g., AEO, D1, D2 and S2). For the first analysis, the invention used all 94 epochs of the basic data set. This set was called “inclusive.” In this approach, the present invention categorized AEC observations with AEO observations. Note that the AEC observations only occurred in mixed states. The present invention also performed statistical tests on a subset of epochs, and their associated behavior states. However, with only a few representatives were eliminated. For this subset, all mixed states were removed. All epochs used in this data set were either purely or predominately one behavior state. This data set was called “restricted.” The total number of epochs that comprise the restricted data was 78 and consisted of 54 interictal and 24 preictal epochs. The qualitative conclusions were the same for both data sets.

To test for the dependence of <Q₂> and <δ₂> on behavior state, on seizure focus location, and on preictal versus interictal, the present invention used a suite of linear statistical models with up to five dummy variables. In this regard, the following (generally binary) variables were introduced:

1) X₁=1 if the behavior state is AEO and X₁=0 otherwise;

2) X₂=1 if the behavior state is D1, and X₂=0 otherwise;

3) X₃=1 if the behavior state is D2, and X₃=0 otherwise;

4) X₄=1 if the seizure focus is on the left side and X₄=0 if the seizure focus is on the right side; and

5) X₅=1 if the epoch is interictal and X₅=0 if the epoch is preictal.

Since the invention used four categories of behavior state, the assignment (X₁,X₂,X₃)=(0,0,0) uniquely corresponds to the behavior state S2. An exception to the assignment of binary values to the X₁, X₂ and X₃ occurred in the characterization of mixed states for the inclusive data set. In this case, the Xi took on values that reflected the fraction of the epoch associated with different behaviors. For example, a mixed state containing 25% of each of the four states, AEO, D1, D2 and S2, was represented by the assignment X₁=X₂=X₃=0.25.

In the case of <Q₂>, for example, the present invention constructed statistical tests for the dependence of <Q2> on the preictal versus interictal states, on the location of the seizure focus (i.e., left or right side of the subject's brain), and on the behavior state (see the following linear relation)

$$〈 {Q}_{2} 〉 = {a}_{0} + {\sum }_{j} {a}_{j} {X}_{j} , \qquad ( Formula 7 )$$

where the aⱼ are real coefficients and the Xⱼ are a subset of the binary state variables defined above. By choosing different subsets of the binary variables, the present invention can test for the dependence of <Q₂> on different behavioral, locational, and temporal states. For example, Formula 7 with a₄=a₅=0, can be used to test for the null hypothesis (i.e., that <Q₂> is independent of behavior state). Similarly, a₁=a₂=a₃=a₅=0 can be set and the null hypothesis tested such that <Q2> does not depend on which hemisphere contains the seizure focus. The present invention can also set a₁=a₂=a₃=a₄=0, to test for the null hypothesis that <Q₂> does not depend on whether the epoch is preictal or interictal. There is no indication that <Q₂> depends on the side of the brain that contains the seizure focus, nor any indication that <Q₂> depends on the behavior state. <Q₂> does depend on whether the epoch in question is preictal or interictal.

The present example also shows that analogous tests applied to the 20 minute averages of the MP's, <δ₂>, imply that there is no dependence on location of the seizure focus, nor on whether the epoch in question is preictal or interictal, but there is dependence on behavior state.

Since <Q₂> or <δ₂> do not necessarily have a linear dependence on state variables, the linear form in Equation 7 was used as a basis to reject the null hypothesis that <Q₂> (or <δ₂>) is independent of various state variables. It is possible that a more complicated form of dependence other than that of Equation 7 would reveal dependencies on state variables if the present approaches failed to reject the null hypothesis. One possibility, for example, is that a finer initial set of behavior states might reveal dependences.

Results for Q₂

To perform the present analyses, the example provides a suite of six linear models of the form Equation 7.
<Q₂>=a₀+a₅X₅ (Test I) (Equation 8)
An estimate of the value of a₅ can be used to test for dependence of <Q₂> on whether the epoch is interictal or preictal. The p-value for the estimate of a₅ is 0.02 when the inclusive data set was used, and is 0.045 when restricted data set was used. P-values less than 0.05 represent a rejection with greater than 95% confidence. Thus, the null hypothesis that a₅=0 can be rejected, which means that the null hypothesis that <Q₂> was independent of whether the epoch in question was preictal or interictal can also be rejected. Moreover, the estimate of a₅ was positive so that <Q₂> for interictal epochs was significantly larger than it was for preictal epochs. This result was consistent with findings that show the dependence of <Q₂> on the temporal proximity of a seizure.

The dependence of <Q₂> on seizure focus (e.g., left or right side of the brain) was tested. Two models were considered:
<Q₂>=a₀+a₄X₄ (Test II) (Equation 9A)
<Q₂>=a₀+a₄X₄+a₅X₅ (Test III) (Equation 9B)
Statistical tests were constructed to reject the null hypothesis that a₄=0. For the model Equation 9A, p-values of 0.22 and 0.24 for the inclusive and restricted data sets, respectively, were found.

In Equation 9B the present invention used the partial F-test to test separately for the null hypotheses that a₅=0 and that a₄=0. It was found that the null hypothesis, a₄=0 (p-value is 0.24 for the inclusive data set and 0.15 for the restricted data set), could not be rejected but that the null hypothesis, a₅=0 (p-value is 0.031 for the inclusive data set and 0.032 for the restricted data set), could be rejected. These results are consistent with dependence of <Q₂> on whether the epoch was interictal or preictal, but not dependent on seizure location.

The present invention also tested the dependence of <Q₂> on behavior state, by considering two additional models:
<Q₂>=a₀+a₁X₁+a₂X₂+a₃X (Test IV) (Equation 10A)
<Q₂>=a₀+a₅X₅+a₁X₁+a₂X₂+a₃X₃ (Test V) (Equation 10B)
In both of these models, the present invention tested the null hypothesis that a₁=a₂=a₃=0. The alternative hypothesis was that at least one of the aⱼ, j=1,2,3 was non-zero. For Equation 10A, the null hypothesis (the p-value is 0.11 for the inclusive data set and 0.13 for the restricted data set) could be rejected. To test the null in Equation 10B, the present invention used the partial F-test, again the null hypothesis could not be rejected (P-values are 0.18 for both the inclusive and restricted data sets). Consequently, <Q₂> does not depend on behavior state.

In another embodiment, the present invention provides a model that incorporates all five state variables simultaneously:

$$〈 {Q}_{2} 〉 = {a}_{0} + {\sum }_{j = 1}^{5} {a}_{j} {X}_{j} ( Test VI ) \qquad ( Equation 11 )$$

The null hypothesis, a₁=a₂=a₃=a₄=0, was tested against the alternative hypothesis that at least one of the aⱼ for j=1,2,3,4 is non-zero. Using both the inclusive and restricted data sets, the partial F test shows that the null hypothesis, (the p-value is approximately 0.22 for each data set) cannot be rejected, and that <Q₂> depends either on behavior state or on the location of seizure focus. The results of the tests for <Q₂> are presented in Table 1.

TABLE 1
Summary of dependence tests for <Q₂>

| | <Q₂> inclusive | | <Q₂> restricted | |
|---|---|---|---|---|
| | data set | | data set | |
| | Null | | Null | |
| | hypothesis | p-value | hypothesis | p-value |
| Test I | Reject | 0.020 | Reject | 0.045 |
| Test II | Accept | 0.22 | Accept | 0.24 |
| Test III | (Accept, | 0.24, | (Accept, | 0.15, |
| | Reject) | 0.031 for | Reject) | 0.032 for |
| | | (a₄, a₅) = 0 | | (a₄, a₅) = 0 |
| Test IV | Accept | 0.11 | Accept | 0.13 |
| Test V | Accept | 0.18 | Accept | 0.18 |
| Test VI | Accept | 0.22 | Accept | 0.22 |

Results for δ₂

The results described above indicate that <Q₂> was not sensitive to a subject's behavior state, but did depend on whether the epoch in question was preictal or interictal. Tests of the adjacent and remote channels using both inclusive and restricted data sets were conducted as follows:
<δ₂>=a₀+a₅X₅ (Test I) (Equation 12)
Null hypothesis a₅=0, alternate hypothesis a₅ 0.
<δ₂>=a₀+a₄X₄ (Test II) (Equation 13)
Null hypothesis a₄=0, alternate hypothesis a₄ 0.
<δ₂>=a₀+a₄x₄+a₅X₅ (Test III) (Equation 14)
Null hypotheses a₄=0 or a₅=0 using partial F-tests.
<δ₂>=a₀+a₁X₁+a₂X₂+a₃X (Test IV) (Equation 15)

Null hypothesis a₁=a₂=a₃=0, alternate hypothesis, at least one of the as (i=1,2,3,) is non-zero
<δ₂>=a₀+a₅X₅+a₁X₁+a₂X₂+a₃X₃ (Test V) (Equation 16)
Null hypothesis a₁=a₂=a₃=0, alternate hypothesis, at least one of the a; (i=1,2,3,) is non-zero.

$$\begin{matrix}Test VI \text{:} & \\ 〈 {δ}_{2} 〉 = {a}_{0} + {\sum }_{j = 1}^{5} {a}_{j} {X}_{j} ( Test VI ) & ( Equation 17 )\end{matrix}$$

Null hypothesis a₁=a₂=a₃=a₄=0, alternate hypothesis, at least one of the as (i=1,2.3.4) is non-zero.

The results for the tested of δ₂ are presented in the following tables.

TABLE 2
Test for dependencies of <δ₂> for
a channel adjacent to the site of ictal onset

| | <δ₂> (adjacent) | | <δ₂> (adjacent) | |
|---|---|---|---|---|
| | inclusive data set | | restricted data set | |
| | Null | | Null | |
| | hypothesis | p-value | hypothesis | p-value |
| Test I | Accept | 0.10 | Accept | 0.27 |
| Test II | Accept | 0.47 | Accept | 0.58 |
| Test III | (Accept, | 0.543, | (Accept, | 0.50, |
| | Accept) | 0.50 for | Accept) | 0.25 for |
| | | (a₄, a₅) = 0 | | (a₄, a₅) = 0 |
| Test IV | Reject | 0.0003 | Reject | 0.0001 |
| Test V | Reject | 0.00002 | Reject | 0.00002 |
| Test VI | Reject | 0.00001 | Reject | 0.00007 |

TABLE 3
Tests for dependencies of <δ₂> for
a channel remote from the site of ictal onset

| | <δ₂> (remote) | | <δ₂> (remote) | |
|---|---|---|---|---|
| | inclusive data set | | restricted data set | |
| | Null | | Null | |
| | hypothesis | p-value | hypothesis | p-value |
| Test I | Accept | 0.77 | Accept | 0.58 |
| Test II | Accept | 0.62 | Accept | 0.64 |
| Test III | (Accept, | (0.77, | (Accept, | (0.60, |
| | Accept) | 0.82) for | Accept) | 0.55) for |
| | | (a₄, a₅) = 0 | | (a₄, a₅) = 0 |
| Test IV | Reject | 0.0001 | Reject | 0.0007 |
| Test V | Reject | 0.00004 | Reject | 0.00003 |
| Test VI | Reject | 0.00002 | Reject | 0.00009 |

These results indicate that, unlike <Q₂>, the individual <δ₂> values from channels adjacent to and remote from the site of ictal onset strongly depend on behavior state.

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A system for predicting ictal onset in a subject comprising:
a. a first data sensor positioned on the scalp of a subject near the focal point of ictal onset;
b. a second data sensor positioned on the scalp of said subject, wherein said second data sensor is remote from said first data sensor; and
c. a processor configured to analyze data collected from said first and said second data sensors to provide a nonlinear mathematical manipulation of said data collected from said first and from said second data sensors, wherein said nonlinear mathematical manipulation produces a first marginal predictability value, and a second marginal predictability value.

2. The system of claim 1, wherein said first and said second data sensors comprise electrodes.

3. The system of claim 2, wherein said electrodes record electroencephalogram data from said subject.

4. The system of claim 1, wherein said processor compares the difference between said first marginal predictability value and said second marginal predictability value.

5. The system of claim 4, wherein said difference between said first marginal predictability value and said second marginal predictability value decreases indicting ictal onset.

6. The system of claim 1, further comprising a subject warning device configured to receive information from said processor.

7. The system of claim 6, wherein said information comprises information predictive of an ictal onset.

8. The system of claim 6, wherein said subject warning device comprises at least one alarm selected from the group consisting of audible, visual, and tactile alarms.

9. The system of claim 1, wherein said processor further comprises a computer readable memory.

10. The system of claim 1, further comprising an anti-seizure agent administering device in communication with said processor wherein said anti-seizure agent administering device administers an anti-seizure agent to the subject.

11. The system of claim 10, wherein said anti-seizure agent administering device is selected from the group consisting if micro pumps and electrical stimuli devices.

12. A method for predicting ictal onset in a subject comprising:
a. providing:
i. a subject;
ii. a system configured to detect ictal onset, wherein said system comprises: a first data sensor positioned on the scalp of said subject near the focal point of ictal onset; a second data sensor positioned on the scalp of said subject, wherein said second data sensor is remote from said first data sensor;
iii. a processor configured to analyze data collected from said first and said second data sensors to provide a nonlinear mathematical manipulation of said data collected from said first and from said second data sensors, wherein said nonlinear mathematical manipulation produces a first marginal predictability value, and a second marginal predictability value; and
iv. a subject warning device in communication with said processor; and
b. contacting said subject with said system;
c. determining said first marginal predictability value and a second marginal predictability value;
d. predicting ictal onset in said patient by difference in said first marginal predictability value and a second marginal predictability value.

13. The method of claim 12, wherein said first and said second data sensors comprise electrodes.

14. The method of claim 12, wherein said electrodes record electroencephalogram data from said subject.

15. The method of claim 12, wherein said processor compares the difference between said first marginal predictability value and said second marginal predictability value.

16. The method of claim 15, wherein said difference between said first marginal predictability value and a second marginal predictability value decreases indicting ictal onset.

17. The method of claim 12, further comprising providing a subject warning device configured to receive information from said processor.

18. The method of claim 17, wherein said information comprises information predictive of an ictal onset.

19. The method of claim 17, wherein said subject warning device comprises at least one alarm selected from the group consisting of audible, visual, and tactile alarms.

20. The method of claim 12, further comprising an anti-seizure agent administering device in communication with said processor wherein said anti-seizure agent administering device administers an anti-seizure agent to the subject.

21. The system of claim 20, wherein said anti-seizure agent administering device is selected from the group consisting if micro pumps and electrical stimuli devices.
